(19) United States
(12) Patent Application Publication
(10) Pub. No.: US 2025/0292507 A1
(43) Pub. Date: Sep. 18, 2025

(54) A SYSTEM AND A METHOD FOR 3D IMAGE PROCESSING, AND A METHOD FOR RENDERING A 3D IMAGE

(71) Applicant: Syngular Technology Limited, Sheung Wan (HK)

(72) Inventors: Yan Sun, Sheung Wan (HK); Kwan Yik Sze, Hong Kong (CN)

(21) Appl. No.: 18/872,301

(22) PCT Filed: Jun. 22, 2022
(86) PCT No.: PCT/CN2022/100501

§ 371 Date: Dec. 6, 2024

(51) Int. Cl. G06T 17/20; G06T 7/00; G06T 15/20

(52) U.S. Cl. CPC G06T 17/20; G06T 7/0012; G06T 15/205; G06T 2207/10081; G06T 2207/10088; G06T 2207/10104; G06T 2207/10116; G06T 2207/30008; G06T 2207/30024

(57) ABSTRACT

A system and method of three-dimensional image processing. comprising the steps of pre-processing at least one set of raw images each including a plurality of two-dimensional source images. wherein each of the two-dimensional source image represents a cross-sectional view of a three-dimensional object at different positions along an axis in a three-dimensional space: and constructing a three-dimensional image representing the three-dimensional object by integrating 3D mesh points extracted from the at least one set of raw images being pre-processed: wherein the three-dimensional image is readable by a first image viewer arrange to render and to facilitate manipulation of the three-dimensional image.

### TECHNICAL FIELD

The present invention relates to a system and a method for processing 3D images, and particularly, although not exclusively, to a system and method for constructing a 3D image or model using a high-performance image processing pipeline.

### BACKGROUND

Augmented reality (AR) or mixed reality (MR) technology may be employed in healthcare and medical applications including providing supplementary information to a surgeon who performs a surgery on a patient. In some example, three dimensional (3D) models or images of the desired target may be displayed to the surgeons or assistants in the operation theatre, or to clinician as a tool for medical diagnostic analysis.

However, the inclusion of these additional images is a complicated process and may require many hours of processing by medically trained persons to prepare the images so as to have the data useful for the surgeon. To ensure the created 3D model is consistent with the actual object/target being manipulated, additional information extracted from medical diagnostic scanning images obtained using various medical imaging systems may need to be processed and combined accurate, in turn increasing the time and resources required to prepare the necessary models before AR or MR may be used in a medical treatment process.

### SUMMARY OF THE INVENTION

In accordance with a first aspect of the invention, there is provided a method of three-dimensional image processing, comprising the steps of pre-processing at least one set of raw images each including a plurality of two-dimensional source images, wherein each of the two-dimensional source image represents a cross-sectional view of a three-dimensional object at different positions along an axis in a three-dimensional space; and constructing a three-dimensional image representing the three-dimensional object by integrating 3D mesh points extracted from the at least one set of raw images being pre-processed; wherein the three-dimensional image is readable by a first image viewer arrange to render and to facilitate manipulation of the three-dimensional image.

In accordance with the first aspect, the plurality of two-dimensional source images includes medical diagnostic scanning images.

In accordance with the first aspect, each of the at least one set of raw images includes a plurality of medical diagnostic scanning images obtained by a selected one of Computed Tomography (CT), Magnetic Resonance Imaging (MRI), Positron Emission Tomograph (PET) and x-ray imaging.

In accordance with the first aspect, the medical diagnostic scanning images are readable by a second image viewer.

In accordance with the first aspect, the step of constructing the three-dimensional image comprises the step of embedding the plurality of two-dimensional source images in the three-dimensional image at corresponding positions along the axis.

In accordance with the first aspect, the first image viewer is arranged to render a cross-sectional view of the three-dimensional object embedding with a two-dimensional view of the cross-section reproduced based on the medical diagnostic scanning image captured at the corresponding position.

In accordance with the first aspect, the two-dimensional view of the cross-section is substantially equal to the corresponding medical diagnostic scanning image read by the second image viewer.

In accordance with the first aspect, the method further comprises the step of mapping the plurality of two-dimensional source images and the positions of the corresponding cross-section in the three-dimensional object along the axis.

In accordance with the first aspect, the method further comprises the step of identifying and segmenting a plurality of components of different attributes or properties in the three-dimensional object.

In accordance with the first aspect, the plurality of components includes bone, soft tissue, fluid or an implant.

In accordance with the first aspect, the three-dimensional object includes at least a portion of an organ, one or more organ, or a combination thereof, of a living organism.

In accordance with the first aspect, the step of identifying the plurality of components comprises the step of facilitating manually masking of one or more of the plurality of components.

In accordance with the first aspect, the step of pre-processing the at least one set of raw images comprises the step of performing an image optimization process to optimize the at least one set of raw images prior to the step of identifying and segmenting the plurality of components.

In accordance with the first aspect, the image optimization process includes an image fusion process arranged to fuse the plurality of two-dimensional source images obtained in different sets of raw images.

In accordance with the first aspect, the image optimization process includes an interpolation process arranged to generate one or more augment images representing the cross-sectional view of the three-dimensional object at a position between two adjacent two-dimensional source images in the corresponding set of raw images captured along an axis.

In accordance with the first aspect, the method further comprises the step of identifying and segmenting different set of raw images obtained by different medical diagnostic scanning methods.

In accordance with a second aspect of the invention, there is provided a method of rendering a three-dimensional image constructed by the method of the first aspect, comprising the step of rendering the three-dimensional image on a display module, wherein the display module is provided with a user interface arranged to facilitate manipulation of the three-dimensional image.

In accordance with the second aspect, the manipulation of the three-dimensional image includes at least one of zooming, rotating, translating the three-dimensional image and/or moving a section plane for sectioning the three-dimensional image along the axis.

In accordance with the second aspect, the step of rendering the three-dimensional image includes overlaying the three-dimensional image on the three-dimensional object when being observed by a user of the display module.

In accordance with the second aspect, the step of rendering the three-dimensional image further comprises the step of identifying one or more anchors being marked on the three-dimensional object.

In accordance with the second aspect, the method further comprises the step of displaying one or more two-dimensional source images selected from the at least one set of raw images and the three-dimensional image simultaneously.

In accordance with the second aspect, the display module includes a head-mounted display module.

In accordance with a third aspect of the invention, there is provided an image processing pipeline comprising: a 2D image pre-processing module arranged to process at least one set of raw images each including a plurality of two-dimensional source images, wherein each of the two-dimensional source image represents a cross-sectional view of a three-dimensional object at different positions along an axis in a three-dimensional space; and a 3D model construction engine arranged to construct a three-dimensional image representing the three-dimensional object by integrating 3D mesh points extracted from the at least one set of raw images being pre-processed; wherein the three-dimensional image is readable by a first image viewer arrange to render and to facilitate manipulation of the three-dimensional image.

In accordance with the third aspect, the plurality of two-dimensional source images includes medical diagnostic scanning images.

In accordance with the third aspect, each of the at least one set of raw images includes a plurality of medical diagnostic scanning images obtained by a selected one of Computed Tomography (CT), Magnetic Resonance Imaging (MRI), Positron Emission Tomography (PET) and x-ray imaging.

In accordance with the third aspect, the medical diagnostic scanning images are readable by a second image viewer.

In accordance with the third aspect, the 3D model construction engine is arranged to embed the plurality of two-dimensional source images in the three-dimensional image at corresponding positions along the axis.

In accordance with the third aspect, the first image viewer is arranged to render a cross-sectional view of the three-dimensional object embedding with a two-dimensional view of the cross-section reproduced based on the medical diagnostic scanning image captured at the corresponding position.

In accordance with the third aspect, the two-dimensional view of the cross-section is substantially equal to the corresponding medical diagnostic scanning image read by the second image viewer.

In accordance with the third aspect, the image processing pipeline further comprises an image mapping module arranged map the plurality of two-dimensional source images and the positions of the corresponding cross-section in the three-dimensional object along the axis.

In accordance with the third aspect, the pre-processing module is further arranged to identify and segment a plurality of components of different attributes in the three-dimensional object.

In accordance with the third aspect, the plurality of components includes bone, soft tissue, fluid or an implant.

In accordance with the third aspect, the three-dimensional object includes at least a portion of an organ, one or more organ, or a combination thereof, of a living organism.

In accordance with the third aspect, the pre-processing module is arranged to receive manual input to facilitate masking of one or more of the plurality of components.

In accordance with the third aspect, the pre-processing module is further arranged to perform an image optimization process to optimize the at least one set of raw images prior to identifying and segmenting the plurality of components.

In accordance with the third aspect, the pre-processing module is arranged to perform an image fusion process so as to fuse the plurality of two-dimensional source images obtained in different sets of raw images.

In accordance with the third aspect, the pre-processing module is further arranged to perform an interpolation process so as to generate one or more augment images representing the cross-sectional view of the three-dimensional object at a position between two adjacent two-dimensional source images in the corresponding set of raw images captured along an axis.

In accordance with the third aspect, the pre-processing module is further arranged to identify and segment different set of raw images obtained by different medical diagnostic scanning systems.

### BRIEF DESCRIPTION OF THE DRAWINGS

Embodiments of the present invention will now be described, by way of example, with reference to the accompanying drawings in which:

FIG. 1 is a schematic diagram of a computer server which is arranged to be implemented as an image processing pipeline in accordance with an embodiment of the present invention;

FIG. 2 is a block diagram showing an image processing pipeline for processing an image in accordance with an embodiment of the present invention;

FIG. 3 is a flow diagram showing process steps in the image processing pipeline of FIG. 2;

FIG. 4A is an example operation in a selected number of process steps performed by the image processing pipeline of FIG. 3;

FIG. 4B is an example operation in a selected number of process steps performed by the image processing pipeline of FIG. 3;

FIG. 4C is an example operation in a selected number of process steps performed by the image processing pipeline of FIG. 3;

FIG. 4D is an example operation in a selected number of process steps performed by the image processing pipeline of FIG. 3;

FIG. 5 illustrates multiple medical diagnostic scanning images showing a cross section of a portion of a human body, before and after denoising and smoothing process performed by the 2D image pre-processing module in the image processing pipeline of FIG. 2;

FIG. 6 illustrates multiple medical diagnostic scanning images showing a cross section of a portion of a human body, before and after an interpolation slice being generated by the 2D image pre-processing module in the image processing pipeline of FIG. 2;

FIG. 7 illustrates a medical diagnostic scanning image showing a cross section of a portion of a human body, before and after a rescaling/resizing process performed by the 2D image pre-processing module in the image processing pipeline of FIG. 2;

FIG. 8A illustrates another set of multiple medical diagnostic scanning images showing a cross section of a portion of a human body, before and after denoising and smoothing process performed by the 2D image pre-processing module in the image processing pipeline of FIG. 2;

FIG. 8B illustrates another set of multiple medical diagnostic scanning images showing a cross section of a portion of a human body, before and after denoising and smoothing process performed by the 2D image pre-processing module in the image processing pipeline of FIG. 2;

FIG. 9A illustrates a medical diagnostic scanning images showing a cross section of a portion of a human body, before a masking process performed by the 2D image pre-processing module using a mask.

FIG. 9B illustrates a mask in the image processing pipeline of FIG. 2;

FIG. 9C illustrates a medical diagnostic scanning images showing a cross section of a portion of a human body, after a masking process performed by the 2D image pre-processing module using the mask in FIG. 9B in the image processing pipeline of FIG. 2;

FIG. 10A illustrates a medical diagnostic scanning image showing a cross section of a portion of a human body, before a bone-edge enhancement process performed by the 2D image pre-processing module in the image processing pipeline of FIG. 2;

FIG. 10B illustrates a medical diagnostic scanning image showing a cross section of a portion of a human body, after a bone-edge enhancement process performed by the 2D image pre-processing module in the image processing pipeline of FIG. 2;

FIG. 11 illustrates 3D images showing 3D model, including an original model, an enhanced intermedia model, and a final model with cinematic effect, of bone structures constructed in accordance with an embodiment of the present invention;

FIG. 12 illustrates a medical diagnostic scanning image showing a cross section of a portion of a human body, before and after a CT/MRI image mapping process performed by the 2D image pre-processing module in the image processing pipeline of FIG. 2;

FIG. 13 is an illustration showing a first-person view of a user wearing a head-mounted display in accordance with an embodiment of the present invention; and

FIG. 14 is illustration showing a first-person view of a user wearing a head-mounted display in accordance with an embodiment of the present invention in a surgical operation.

FIG. 15 is illustration showing a first-person view of a user wearing a head-mounted display in accordance with an embodiment of the present invention in a surgical operation.

### DETAILED DESCRIPTION OF THE PREFERRED EMBODIMENT

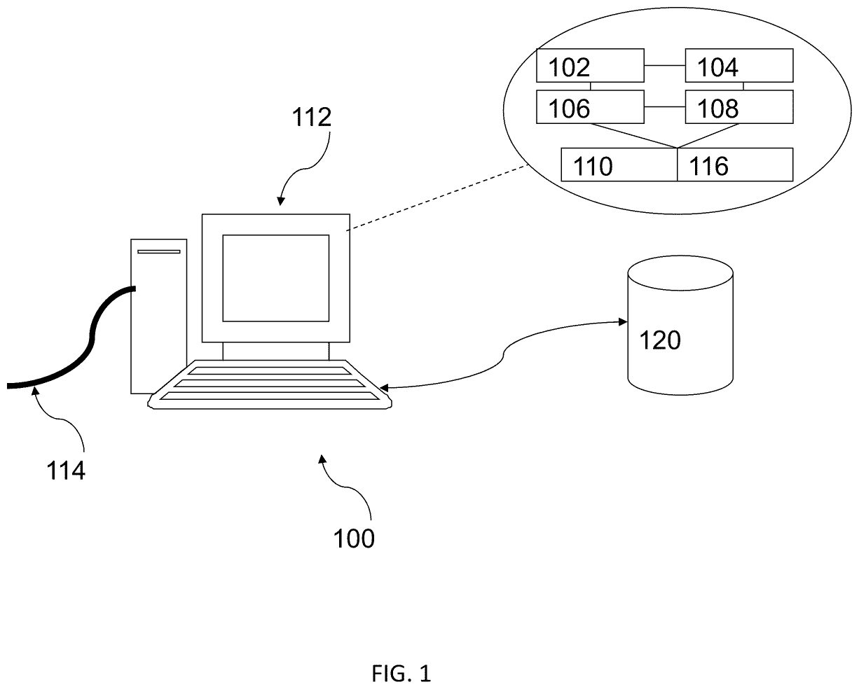

Referring to FIG. 1, an embodiment of computer system which may be implemented to function as a system for 3D image processing in accordance with an aspect of the present invention is illustrated. This embodiment of the system for 3D image (or 3D model or 3D object) processing is arranged to provide an image processing pipeline comprising: a 2D image pre-processing module arranged to process at least one set of raw images each including a plurality of two-dimensional source images, wherein each of the two-dimensional source image represents a cross-sectional view of a three-dimensional object at different positions along an axis in a three-dimensional space; and a 3D model construction engine arranged to construct a three-dimensional image representing the three-dimensional object by integrating 3D mesh points extracted from the at least one set of raw images being pre-processed; wherein the three-dimensional image is readable by a first image viewer arrange to render and to facilitate manipulation of the three-dimensional image.

In this example embodiment, the system for processing a 3D image may include an interface and processor which are implemented by a computer having an appropriate user interface. The computer may be implemented by any computing architecture, including portable computers, tablet computers, stand-alone Personal Computers (PCS), smart devices, Internet of Things (IOT) devices, edge computing devices, client/server architecture, “dumb” terminal/mainframe architecture, cloud-computing based architecture, or any other appropriate architecture. The computing device may be appropriately programmed to implement the invention.

The system may be used to process medical images to generate a 3D image (which for the purpose of this document, the term 3D image includes 3D models or 3D objects such as 3D graphical objects) that may be provided to users, such as medical experts or practitioners to review or otherwise manipulate. The system is arranged to process one or more medical imaging sources, such as those as captured by existing medical imaging tools (Ultra-sounds, X-rays, MRI, CT Scans, etc) and process these images with automated or semi-automated processes to produce a 3D model that may be presented, viewed, reviewed or manipulated by a user. The systems may be advantageous as users, such as medical practitioners, may be able to use the system to create a unique 3D image or model with less effort, resources or time, and also to use the 3D image or model for manipulation or review either before or during a procedure, and thus improving the quality of the medical treatment provided.

As shown in FIG. 1 there is a shown a schematic diagram of a computer system or computer server 100 which is arranged to be implemented as an example embodiment of a system for processing an image. In this embodiment the system comprises a server 100 which includes suitable components necessary to receive, store and execute appropriate computer instructions. The components may include a processing unit 102, including Central Processing United (CPUs), Math Co-Processing Unit (Math Processor), Graphic Processing United (GPUs) or Tensor processing united (TPUs) for tensor or multi-dimensional array calculations or manipulation operations, read-only memory (ROM) 104, random access memory (RAM) 106, and input/output devices such as disk drives 108, input devices 110 such as an Ethernet port, a USB port, etc. Display 112 such as a liquid crystal display, a light emitting display or any other suitable display and communications links 114. The server 100 may include instructions that may be included in ROM 104, RAM 106 or disk drives 108 and may be executed by the processing unit 102. There may be provided a plurality of communication links 114 which may variously connect to one or more computing devices such as a server, personal computers, terminals, wireless or handheld computing devices, Internet of Things (IoT) devices, smart devices, edge computing devices. At least one of a plurality of communications link y be connected to an external computing network through a telephone line or other type of communications link.

The server 100 may include storage devices such as a disk drive 108 which may encompass solid state drives, hard disk drives, optical drives, magnetic tape drives or remote or cloud-based storage devices. The server 100 may use a single disk drive or multiple disk drives, or a remote storage service 120. The server 100 may also have a suitable operating system 116 which resides on the disk drive or in the ROM of the server 100.

The computer or computing apparatus may also provide the necessary computational capabilities to operate or to interface with a machine learning network, such as neural networks, to provide various functions and outputs. The neural network may be implemented locally, or it may also be accessible or partially accessible via a server or cloud-based service. The machine learning network may also be untrained, partially trained or fully trained, and/or may also be retrained, adapted or updated over time.

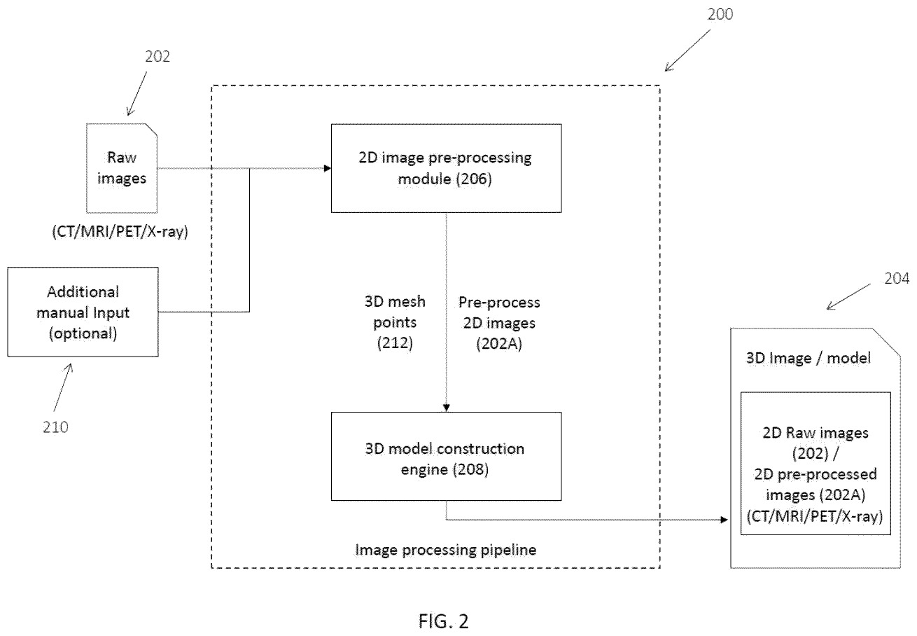

With reference to FIG. 2, there is shown an embodiment of the system 200, for example an image processing pipeline, for processing an image. In this embodiment, the server 100 is used as part of a system 200 as arranged to receive RAW images 202, such as multiple 2D CT or MRI scans or a target object or portion of a target, process these input images 202 such as by denoising and applying filters on these images 202, and finally generate a 3D model 204 incorporating with the 2D images 202 (or processed images 202A) of the target object, such as an organ of a living organism.

Preferably, the system 200 may automatically optimize the input images 202 which may be viewed by using a second image viewer, such as DICOM viewer designed for opening 2D medical diagnostic scanning images of different formats, and construct a 3D image or model 204 which may be viewed and manipulated by a surgeon or a medical practitioner using a first image viewer, such as a viewer app installed in a display apparatus which may be used in an operation theatre, with the possibility of allowing observing of the original 2D medical diagnostic scanning images as if the 2D scanning images or the raw images 202 are viewed by using the second image viewer or a DICOM viewer.

For example, a set of Magnetic Resonance Imaging (MRI) scans showing cross-sectional view of a liver of a patient at different positions along a predetermined axis may be provided to the image processing pipeline as input RAW images, after these 2D scans are obtained from the MRI scanning system. The image processing pipeline may identify multiple 3D mesh points 212 representing a feature point of a 3D model representing the liver by extracting the points from the RAW images, and by integrating all the 3D mesh points 212, the image processing pipeline may construct a 3D model of the liver for further observation by a surgeon, e.g., before or during a surgical operation performed on the patient.

Alternatively, other types of medical diagnostic scanning images obtained by Computed Tomography (CT), Positron Emission Tomograph (PET) or x-ray imaging may be used as input, according to different applications or characterizations of the target organ or object. For example, soft tissues may be better captured by using MRI, whilst CT may work better for capturing tissues with a higher density (such as bone). These medical diagnostic scanning images may be viewed or opened by 2D image viewers such as a DICOM viewer designed for opening these medical diagnostic scanning images.

Without wishing to be bound by theory, medical diagnostic scanning images may be obtained as “slices” by taking multiple cross-sectional images of a body of a living organism such as a human being along a desired axis at a predetermined distance interval, and therefore a three-dimensional image of the portion of the body may be generated by concatenating or stacking multiple cross-sectional images. It should be appreciated that the quality of the generated 3D image may be enhanced by increasing the resolution of the source 2D images and the number of slices in the set medical diagnostic scanning images.

Preferably, the generated 3D model 204 or image may be readable by a dedicated image viewer which is designed for opening, viewing, and manipulating the 3D image. In one example embodiment, a display module such as a head-mounted display (HMD) module may be worn by a surgeon when he performs a surgical operation, and the HMD may be installed with such image viewer (the first image viewer) for viewing and manipulating, such as zooming, rotating, translating the three-dimensional image and/or moving a section plane for sectioning the three-dimensional image along the axis the 3D model 204. It should be appreciated by a skilled person that the 3D image may be alternatively displayed or rendered on other display modules or devices.

Preferably, the two-dimensional view of the cross-section is substantially equal to the corresponding medical diagnostic scanning image read by the second image viewer. 2D view of the original 2D medical diagnostic scanning images may be simultaneously observed when using the dedicated image viewer for viewing the 3D image, for example, when the surgeon is viewing a cross-sectional view of the 3D model of the object using the HMD, the corresponding 2D medical diagnostic scanning image may be presented to the surgeon and/or other observer, without needing to using a separate DICOM viewer to read the 2D RAW images.

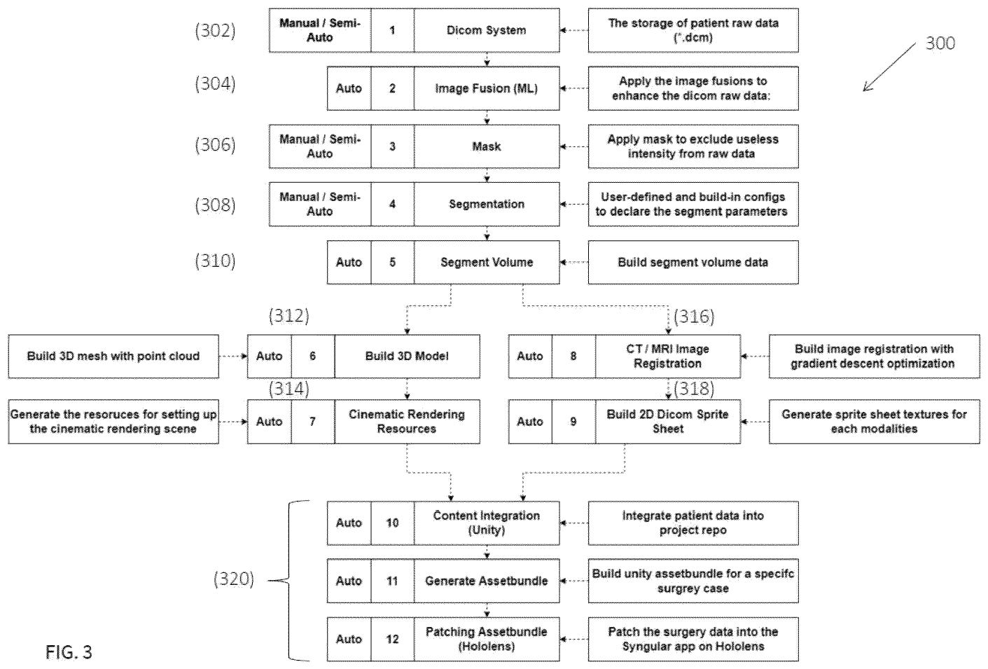

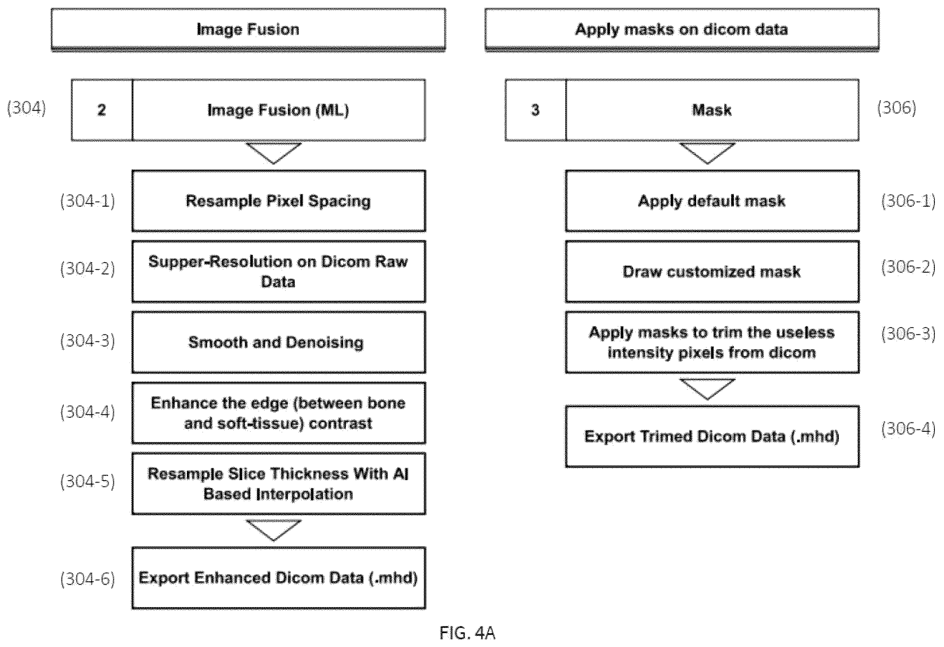

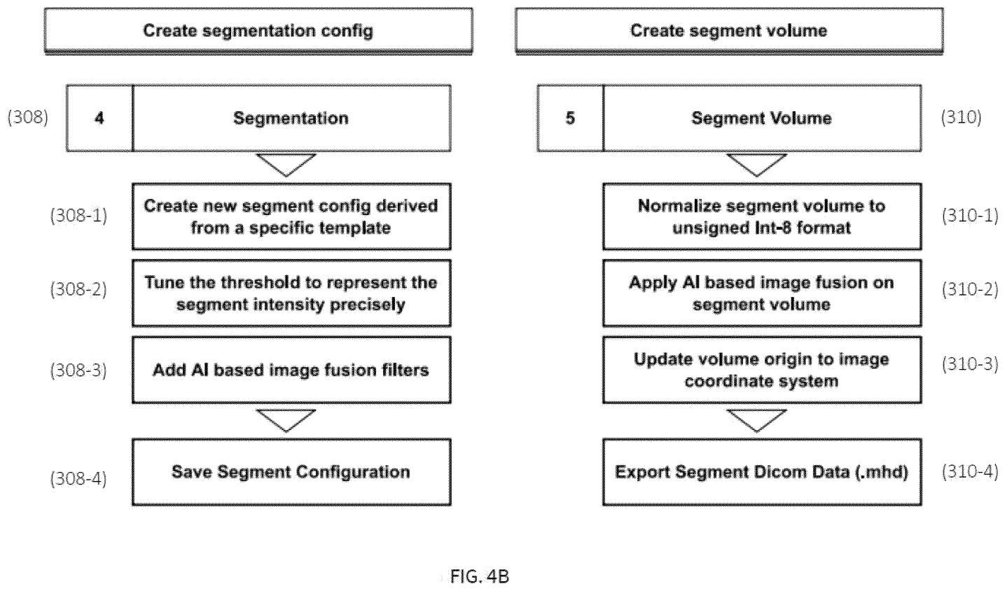

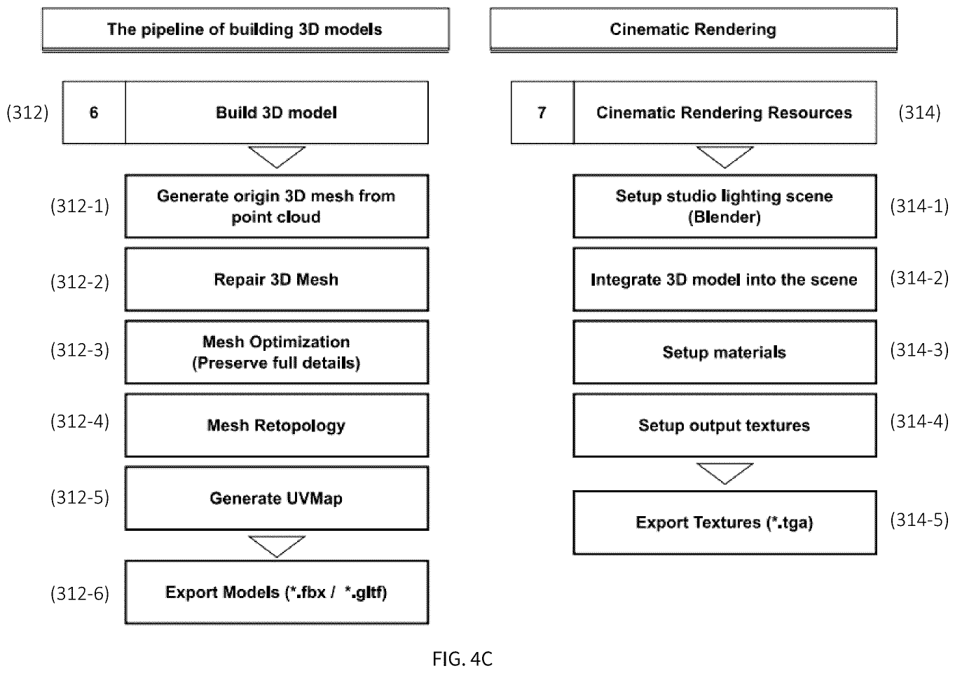

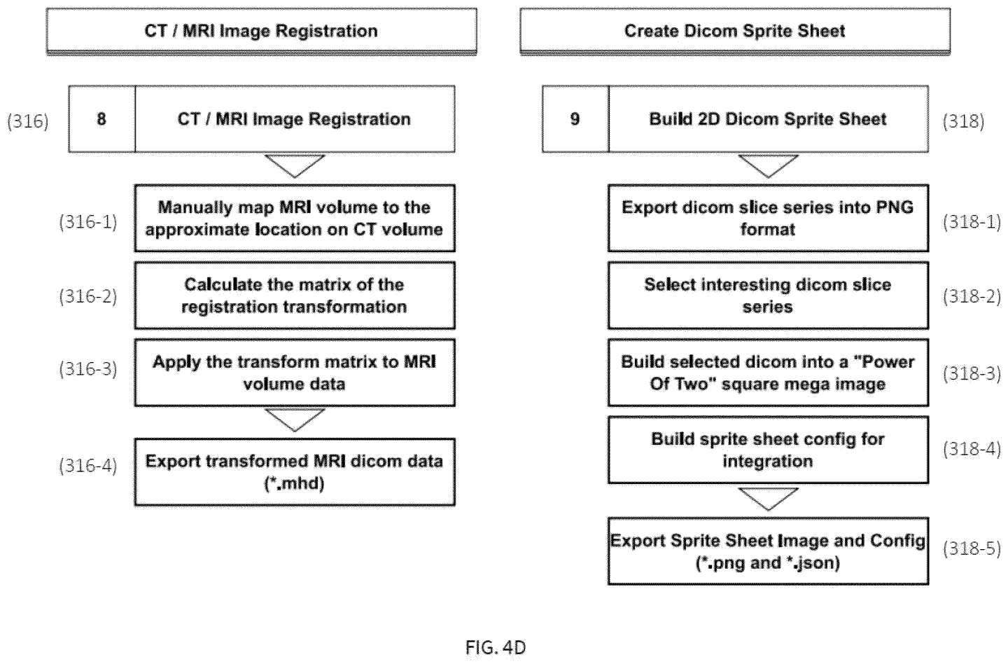

With reference also to FIG. 3 and FIGS. 4A to 4D, the processing method 300 involved in the image processing pipeline includes a number of steps to process the 2D RAW images 202 before and after generating the 3D model 204 or image of the object of interest. Preferably, the 2D image pre-processing module 206 may optimize the plurality of images 202 received by the pipeline using one or more pre-process so as to improve the accuracy of the 3D mesh points generated for 3D model constructions.

For example, the process 300 may start with, at step 302, providing a plurality of medical diagnostic scanning images which may be referred as the DICOM system that comprises one or more sets of CT/MRI/PET/X-ray scans that may be viewed and observed by a medical practitioner using a DICOM viewer (the second image viewer). The medical diagnostic scanning images 202 may be obtained by capturing new scans using suitable medical imagine equipment or retrieved from a database. This process may be manual or semi-automatic as it may require an operator to specify which set of images should be provided for further process. DICOM is a standard format for different image modalities to be read and process within the same software platform, and the DICOM system may provide a robust infrastructure for DICOM Modality Worklist that can effectively scale with growth and provide a highly compatible solution for adapting to different hospitals.

Once the RAW images 202 have been provided, the 2D image pre-processing module 206 may perform an image optimization process to optimize the raw images 202, which may be useful to improve the later steps of identifying and segmenting a plurality of components in the 2D images 202.

Preferably, the pre-processing module 206, at step 304, may perform an optional image fusion process so as to fuse the plurality (when available) of two-dimensional source images obtained in different sets of raw images. For example, two different sets of RAW images 202 obtained by CT and MRI may be provided to the system 200 for 3D image construction.

For example, patients may take medical scans across different modalities, different time, different postures and perhaps with different scanning protocols among different technicians and organization standards. As a result, there is a very large variation even within the same clinical case, however, for clinicians to accurately diagnose and plan a case, they may need to look across different image scans. Preferably, an accurate fusion of different images would greatly facilitate the understanding of the pathology.

The inventor devised that it may be straightforward to restore high quality anatomical information from high resolution raw data, but the challenging part is to handle the highly variable noise generated from different types of raw images and subsequently reconstruct a high-quality 3D anatomical model.

Preferably, the image pre-processing module 206 may further perform an AI-assisted image fusion process for enhancing DICOM raw data and subsequent 3D reconstruction process. The image fusion process may include the following steps, including the preferred steps of resampling pixel spacing (step 304-1), supper-resolution on DICOM Raw Data (step 304-2), smoothing and denoising (step 304-3), enhancing the edge (e.g., between bone and soft-tissue) contrast (step 304-4), resampling slice thickness with AI based interpolation (step 304-5) and finally exporting enhanced DICOM data (step 304-6).

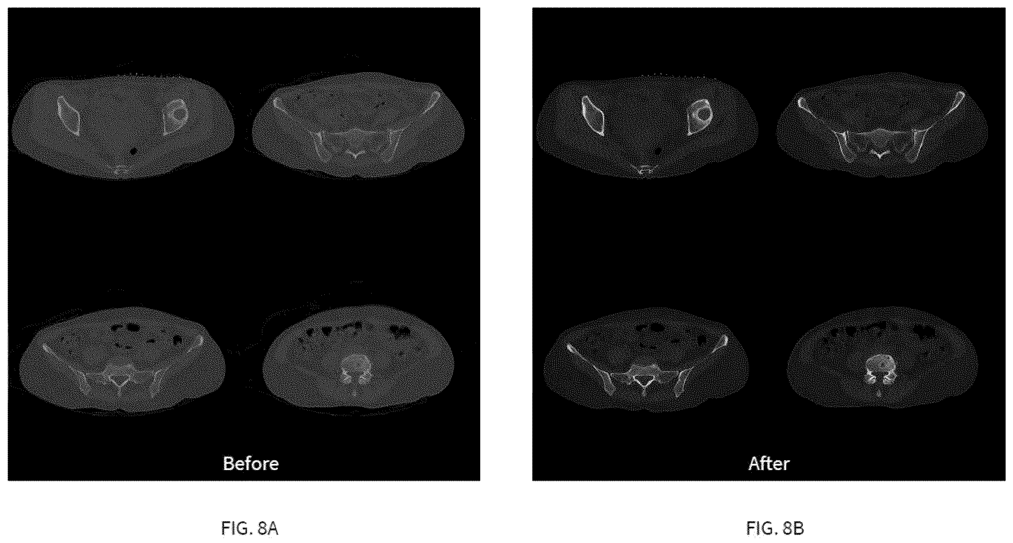

Preferably, AI-based medical image denoising may adapt different strategies for both CT and MR images. Augment bone and soft tissue contrast by AI based edge-enhancing technique. With reference also to FIG. 5, noise in the RAW images on the left side of the Figures has been removed so the features rendered in the denoised image is clearer. Another set of original RAW image and pre-processed image obtained after denoising and smoothing is illustrated in FIGS. 8A and 8B respectively, the scanned features become more observable by the naked eye, and is therefore also more easily become recognizable by an image processor as the edges of the features are clearer (or with a higher contrast), thus 3D mesh points extracted based on these edges may be more accurate.

In addition, the pre-processing module 206 may further perform an interpolation process so as to generate one or more augment images representing the cross-sectional view of the three-dimensional object at a position between two adjacent two-dimensional source images in the corresponding set of raw images captured along an axis.

With reference to FIG. 6, AI-based interpolation techniques can help increase the number of raw data slices, e.g., for creating an interpolation slice 606 representing a cross-sectional image at a position between two adjacent scans 602 and 604, i.e., slice—001 and slice 002, at a 3 mm distance along the axis. This process approximates adding richer point cloud data to provide higher accuracy data for the later process of 3D surface reconstruction, thus generating higher-quality model data, by adding another layer of 3D mesh points at the position between the original slices separated by 6 mm initially.

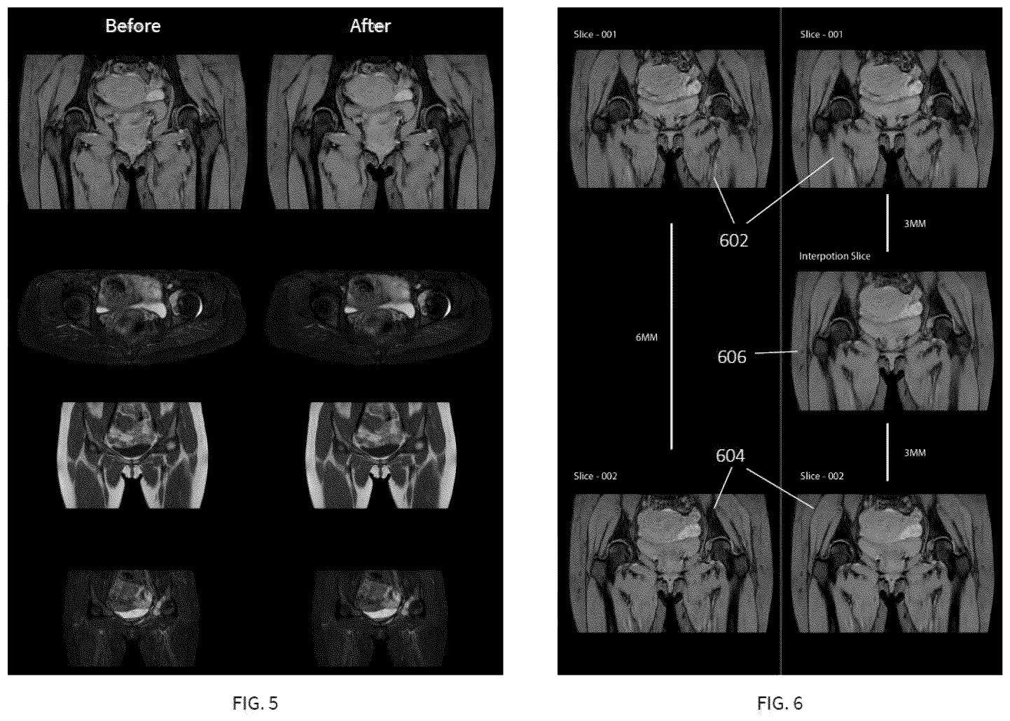

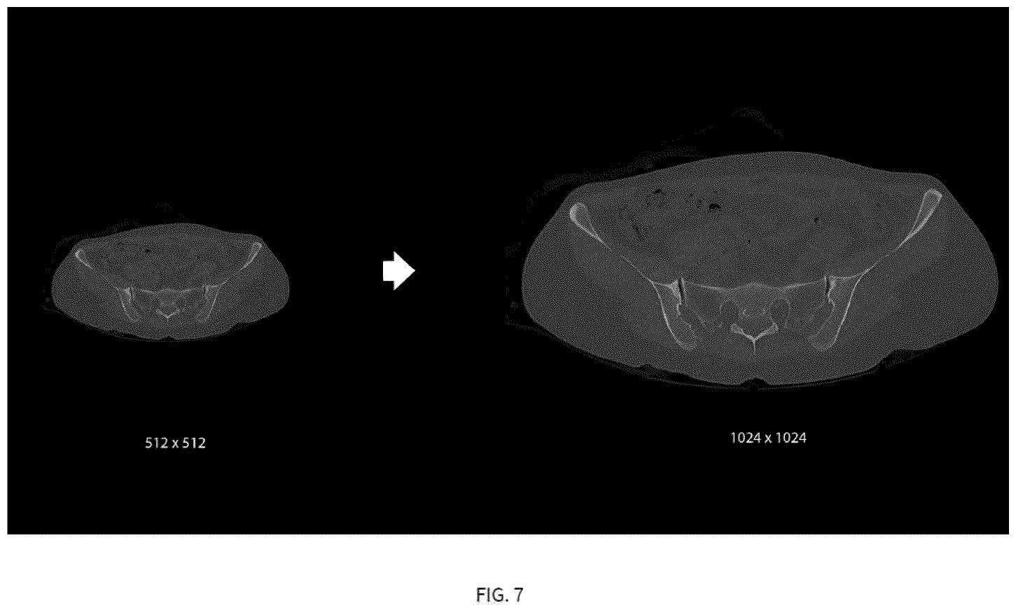

Optionally or additionally, the pre-processing module 206 may further resample or rescale the input RAW images 202, such that the 3D model construction engine 208 may process all RAW images with the same size and resolution. For example, a GAN-based image super-resolution technique may be employed to upscale an original RAW image with a resolution of 512 by 512 to an output image with a resolution of 1024 by 1024 for further processing, referring to FIG. 7. In addition, the pre-processing module may resample DICOM raw data into standard pixel spacing system (e.g., convert pixel spacing 0.617578125 mm×0.617578125 mm to 0.5 mm×0.5 mm). Advantageously, uniform data standards will facilitate the registration of CT and MRI based on image-sensitive areas to achieve a higher accuracy in the constructed 3D model 204.

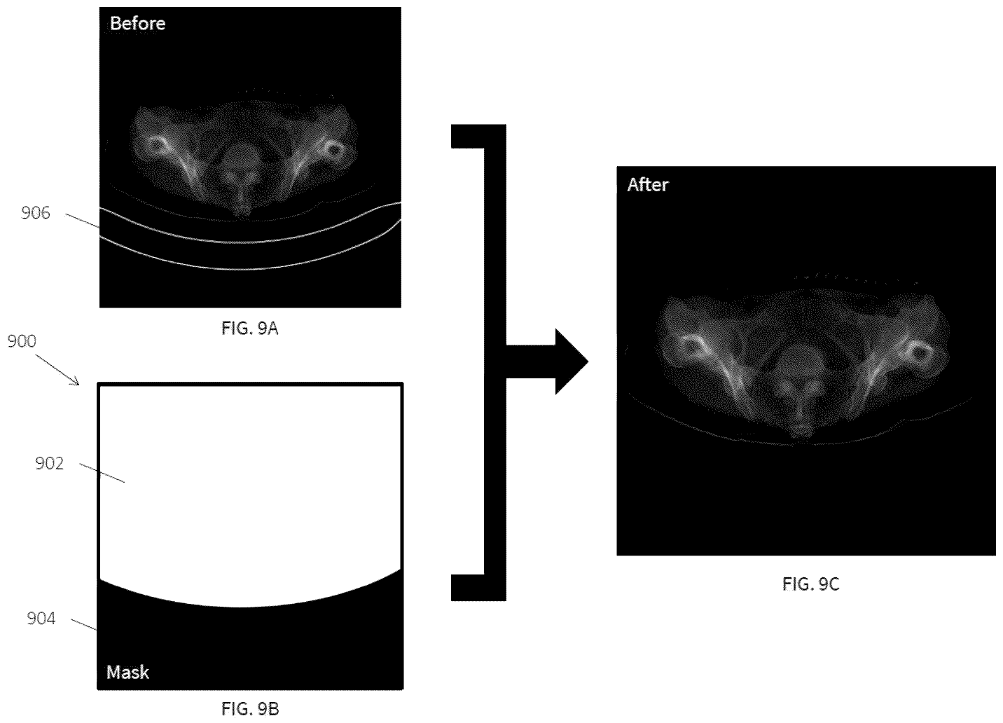

After the image fusion process, at step 306, the pipeline may perform a masking process to exclude any useless intensity from raw data. With reference to FIG. 9A to 9C, a mask 900 which identify a “useful” component/region/portion 902 and a “useless” component region/portion 904 may be provided to the image processing pipeline, such as by receiving a manual input 210 (drawing customized mask) to facilitate masking of one or more of the plurality of components, e.g., by excluding a medical implant 906 being captured by the scanned images. Since adjacent scans or slides may including the same or very similar useful and useless components in each of the scans, additional masks for excluding or eliminating useless components for the other 2D images in the same set of raw images may be automatically generated, with reference to a first mask generated based on manual input 210.

Preferably, the masking step 306 may include the following steps, including applying a default mask (step 306-1), drawing customized mask (step 306-2), applying masks to trim the useless intensity pixels from DICOM (step 306-3) and finally exporting trimmed DICOM data (step 306-4).

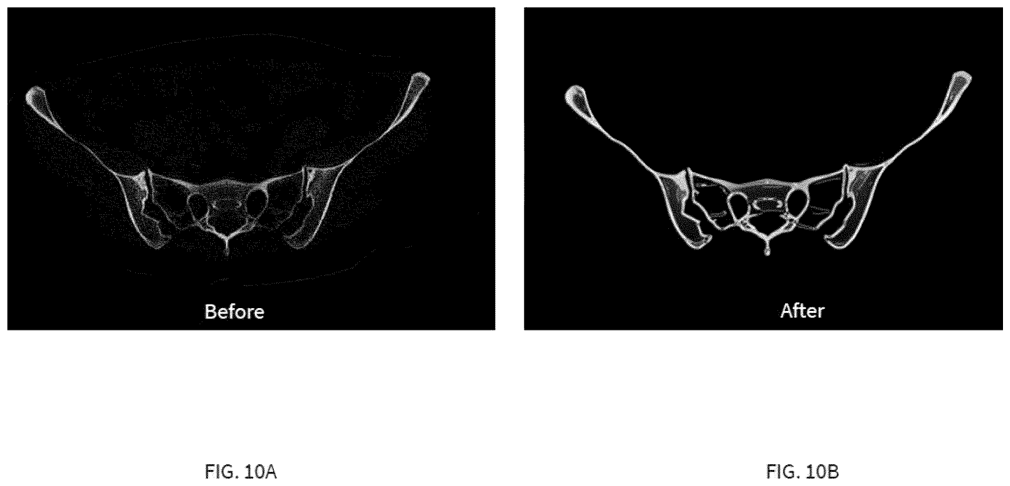

To further improve the 3D mesh points 212 generated based on the shape of objects or components of interests, at step 308, the images 202 may be further optimized using other image processing techniques such as edge enhancement, as edges outlining different features or components in a plurality of images may help the process to identify and segmenting different components in a single image or a set of images representing the cross-sections of a portion of a patient at different positions along an axis. For example, referring to FIG. 10A and 10B, a bone-edge enhancement has been applied to “highlight” the bone components in the obtained image, in that case, the system will be more readily segmenting bone from soft tissues in the same images, as well as the other images in the same set of images.

Preferably, the segmentation step 308 may include the follows steps, including creating new segment configurations derived from a specific template (step 308-1), tuning the threshold to represent the segment intensity precisely (step 308-2), adding AI based image fusion filters (step 308-3) and finally saving segment configuration (step 308-4).

In an alternative example, other user-defined and/or build-in configs may be used to declare the segment parameters, so as to segment or separate different components such as internal organs, muscle, fat, blood vessels, nerve, etc. from other components in the raw image. The segment parameters or configurations may be derived from a template stored in a database, and the operator may tune the threshold of each of the components to represent the segment intensity precisely, to selectively show or hide the unwanted components if necessary. In addition, AI-based image fusion filters may also be applied in this segmentation step 308.

It should be appreciated by a skilled person that manual input may not be necessary in every step or processed in the pipeline, e.g., if the AI-based processing engine is well-trained with sufficient training examples. In the present example with reference to FIGS. 2 and 3, processed such as providing the raw data, masking of useless components in the image, and segmentation based on one or more user definition may be identified as “manual” or “semi-auto” process as it may require at least some user input to further improve the efficiency or accuracy of the constructed 3D image. However, it is also possible that these steps or process may be implemented as fully automated.

To construct a 3D model based on multiple 2D cross-sectional images at different positions of the 3D object, at step 310, volumes of different segments of are required to be assigned or updated, in accordance with the origin to the image coordinate system. At step 310-1, segment volume is normalized to unsigned Int-8 format, at step 310-2, AI based image fusion may be applied on segment volume, then at step 310-3, volume origin is updated to image coordinate system, and finally at step 310-4, segment DICOM data (i.e., volume/mesh data) may be exported and further used for 3D model generation.

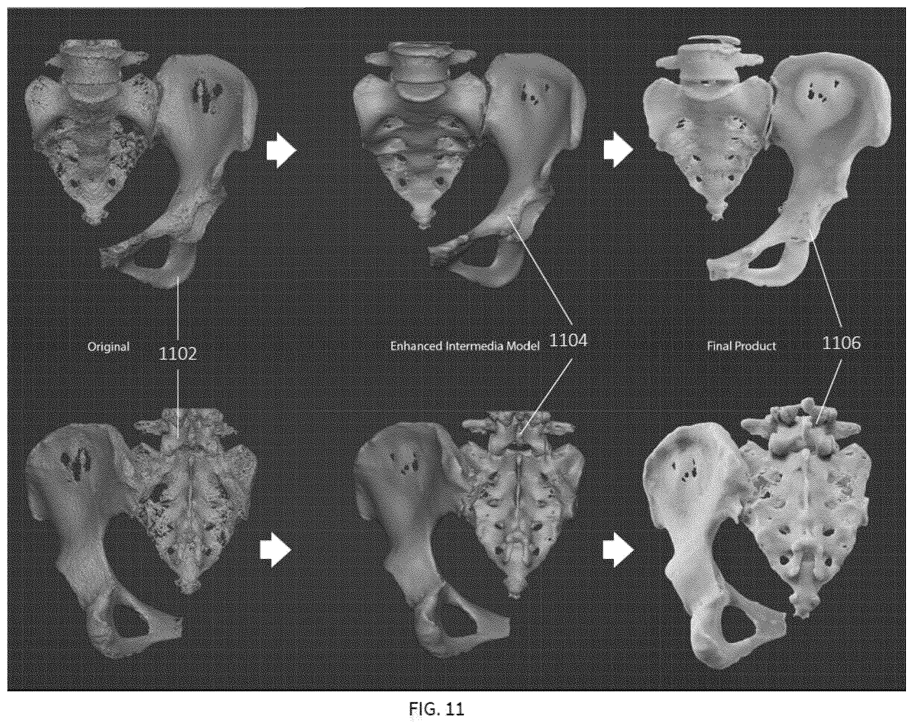

With the exported segment volume data, origin 3D mesh from point cloud may be generated at step 312, and then a 3D model/image 204 may be constructed by integrating the generated 3D mesh points 212 extracted from the raw images 202 being pre-processed by the 2D image processing module 206 as described above, as shown in FIG. 11 which illustrates the bone structures 1102 of a patient near the hip of a human being. Optionally to further improve the visual representation of the 3D image 1102 constructed based on 3D point cloud at step 312-1, one or more optimization method such as repairing the 3D mesh at step 312-2, mesh optimization at step 312-3, mesh retopology at step 312-4 or UV map generation at step 312-5 may be applied, to improve the surface and edges of the 3D image generated by connecting the 3D mesh points together, such that these edges or surfaces may be less corrugated when being viewed and/or manipulated by a clinician, as shown in the “enhanced intermedia model” which may be more preferred by surgeons or medical practitioner as the enhanced model includes much less unnecessary textures. Finally, the 3D model 1104 (as illustrated in FIG. 11) may be exported and saved at step 312-6.

In addition, at step 314, cinematic rendering filter may be applied to further enhance the representation of the 3D image 204, so as to further improve the visual appearance of the 3D model created. For example, a studio lighting scene (a.k.a. blender) may be setup at step 314-1, then the constructed 3D model may be integrated into the scene at step 314-2. By setting up the materials at step 314-3 and applying the material texture on the surface of the 3D model at step 314-4, the final 3D model 1106 as shown in FIG. 11 may be more visually appealing when being viewed by the medical practitioner or the surgeon. The texture data may be exported at step 314-5. It is also possible that lighting condition may be set in the viewer such that the directions and/or number of light sources may be altered so as to further change the appearance of the 3D image representing the 3D object of interest, as shown in the “final product” 1106 in FIG. 11.

As described earlier, multiple set of medical diagnostic scanning images may be provided as two-dimensional source images for 3D image construction, it is possible that the position/volume of segments from different scans may be different, therefore may introduce some shift or offsets in the image representation. Preferably, the method comprises, at step 316, an image mapping or registration process which allow manually mapping or the registering of MRI volume to the approximate location on CT volume at step 316-1, then the image mapping module may map the plurality of two-dimensional source images and the positions of the corresponding cross-section in the three-dimensional object along the axis, by calculating the matrix of the registration transformation at step 316-2 and applying the transform matrix to MRI volume data at step 316-3. Finally, the transformed MRI DICOM data may be exported at step 316-4.

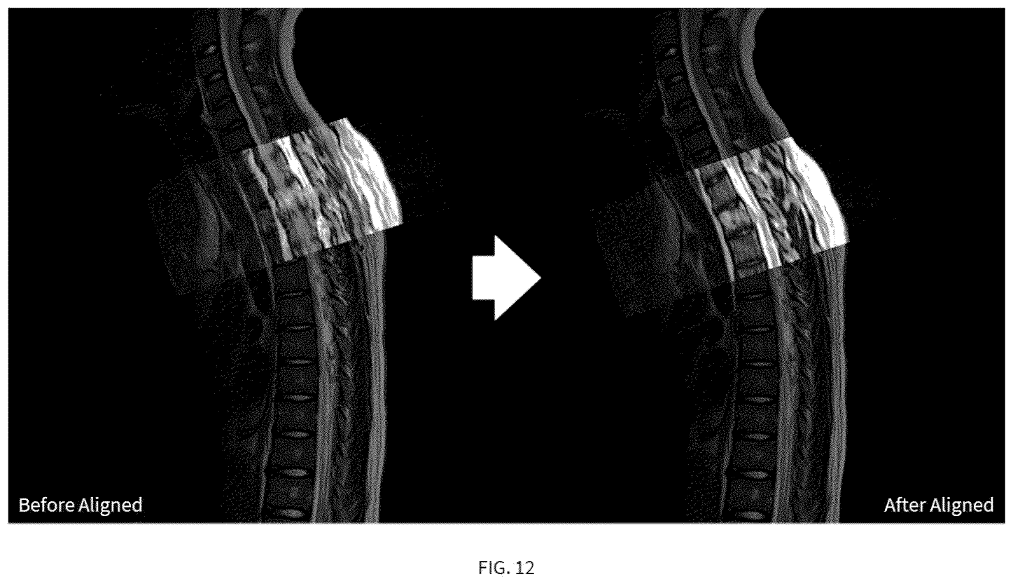

Referring to FIG. 12, the MRI segment and the CT segment are aligned such that the presented cross-section does not produce any misalignment between two source images, when a cross-sectional image of a 3D model at a selected position is presented to the observer. Alternatively, the mapping process may be performed by manually mapping the CT volume against the MRI volume, so as to align the CT and MRI (and/or other sets of source images) related to the same 3D model.

Preferably, the image viewer installed in the HMD may render a cross-sectional view of the three-dimensional object embedding with a two-dimensional view of the cross-section reproduced based on the medical diagnostic scanning image captured at the corresponding position, and optionally, displaying also the source images selected from the at least one set of raw images and the three-dimensional image simultaneously, such that the surgeon, or clinician may simultaneously view the 2D images not different from viewing the medical diagnostic images using the DICOM viewer.

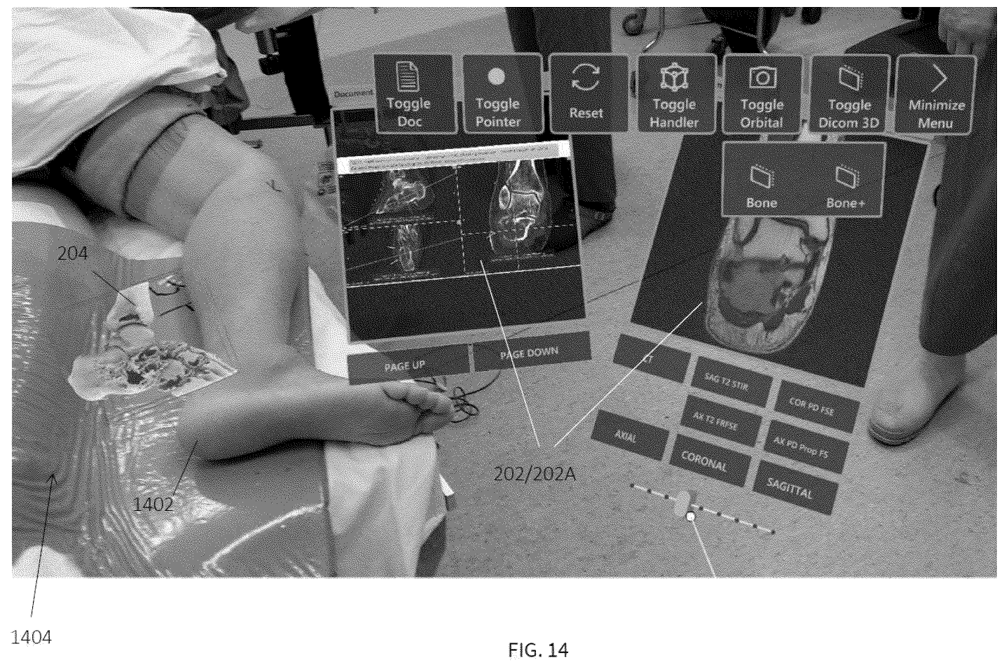

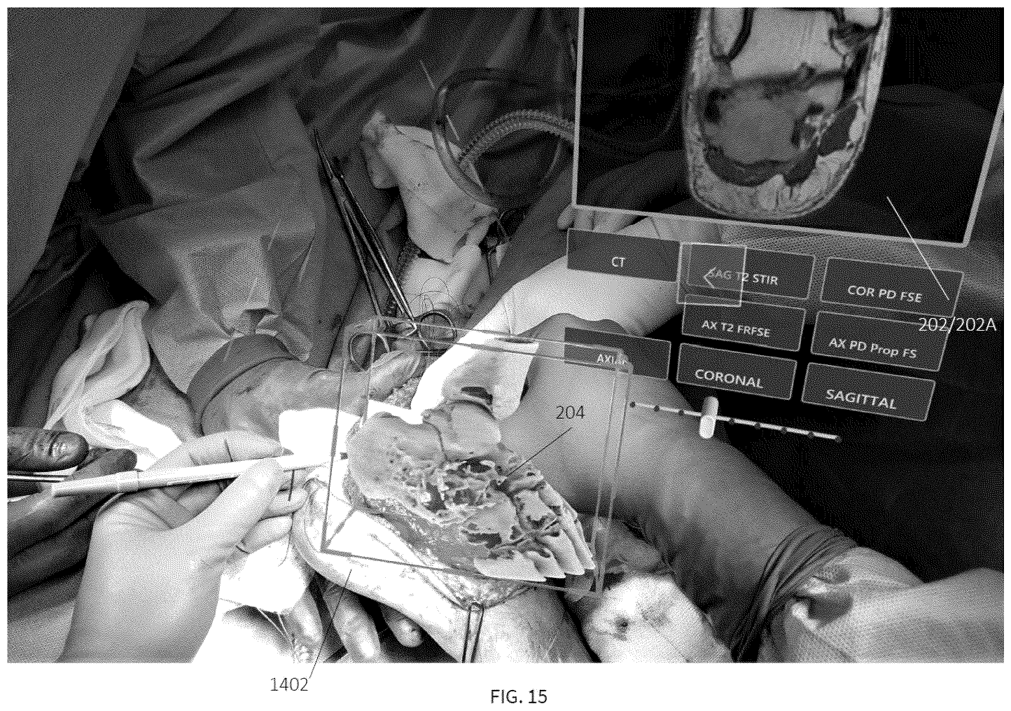

In one example embodiment, at step 318, a DICOM sprite sheet may be built, by exporting the DICOM slice series into PNG format at step 318-1, therefore it is not necessary to use a DICOM viewer to open the DICOM slices such as CT/MRI scans. The clinician or the surgeon may also select only interesting DICOM slice series or all slices in different positions along an axis of the 3D model at step 318-2. Then, selected DICOM maybe built into a “power of two” square mega image at step 318-3. Example rendering showing both cross-sections and interested DICOM slices are illustrated in FIGS. 14 and 15 which will be described later. Preferably, at step 318-4, a sprite sheet associating the PNG files and the 3D model may be built and stored as a configuration file which may be referred when the 3D image viewer needs to locate and open the matching PNG file when the clinician is viewing the corresponding cross-sectional view of the 3D image. Finally, at step 318-5, the sprite sheet image and configuration are exported.

Optionally, the image processing pipeline may further include additional steps 320 for recording information associated with the patent such that the file data structure or package may contain all necessary information related to a surgical operation, such as patient data and operation theatre information, together with the constructed 3D image and the source 2D images reformatted in PNG format and the mapping table, packaged in an “assetbundle” which may be saved in the local storage of a HMD device. For example, the assetbundle or the surgical data may be patched into a Syngular app on a Hololens device so that a surgeon can review any of the abovementioned information when necessary, during a surgical operation.

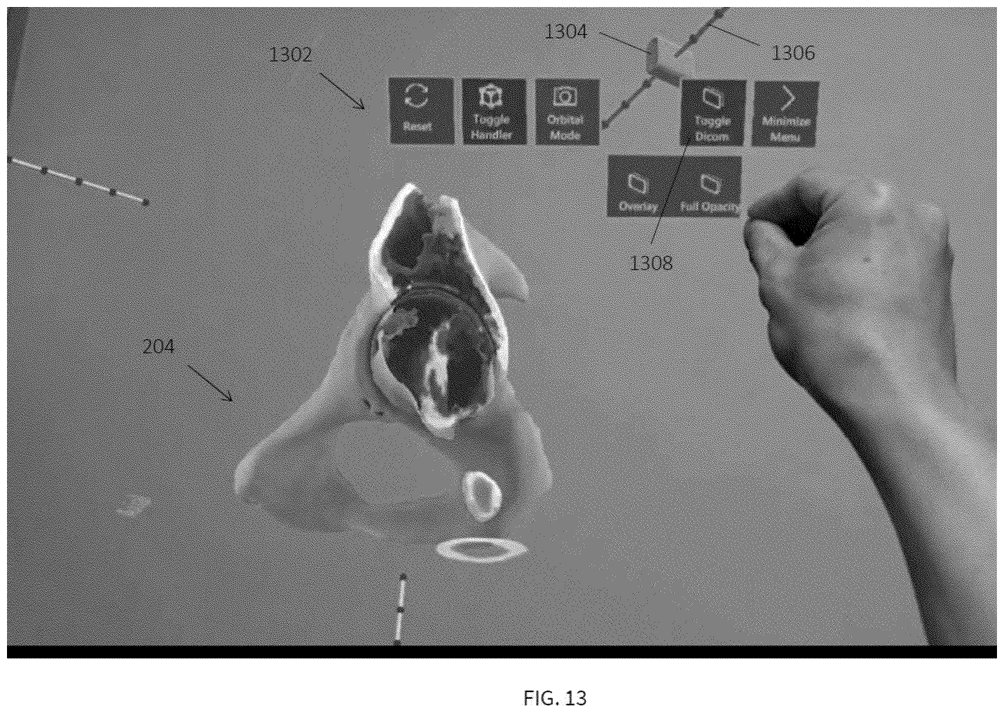

With reference to FIG. 13, there is shown an example operation of an HMD arranged to display or render a three-dimensional image 204 constructed by the image processing pipeline 200 described earlier. In this example, the display module is also provided with a user interface 1302 arranged to facilitate manipulation of the three-dimensional image 204.

Preferably, the 3D image 204 is render at a centre position of the screen of the head mounted display apparatus, and the user may manipulate the three-dimensional image by performing different gestures or interacting with the user interface 1302. The manipulation may include at least one of zooming, rotating, translating the three-dimensional image and/or moving a section plane for sectioning the three-dimensional image along the axis. For example, the user may “grab” the control tab 1304 positioned on the axis 1306 and move the tab to different position along the axis 1306 to move the section plane, and then releasing the tab 1304 to stop the section plane at the selected position. In this configuration, the cross-section embedded in the 3D image 204 at the selected position is displayed to the user. Alternatively, the 3D image 204 may be manipulated by performing a grab gesture directly on the 3D model 204 and then rotating the model 204 by twisting the wrist.

Optionally, the user may “tab” the “toggle DICOM” button 1308 to change to view to “2D view” and read the 2D cross-sectional images 202 using a DICOM viewer or other suitable 2D image viewer. Alternatively, the HMD may display one or more two-dimensional source images 202 selected from the at least one set of raw images, by manipulating the 3D image 204 directly, and the 3D image 204 simultaneously.

In this example, the 3D image 204 is constructed by providing to the image processing system a set of CT or MRI scans (raw images 202) of the bone structures of interest, then the image processing pipeline 200 automatically processes all the sources 2D images 202 and creates the 3D model 204 integrated (or embedded) with the associated cross-sectional scans 202/202A.

With reference to FIGS. 14 and 15, there is shown an alternative example operation in which the images represent a first-person view of a surgeon who performs a medical operation to an ankle 1402 of a patient 1404. In this example, the HMD overlays the three-dimensional image 204 on the three-dimensional object 1402 when being observed by a user of the display module, however, the 3D image/model 204 is not aligned with the actual position of the 3D object, i.e., the ankle 1402 of the patient 1404, as shown in FIG. 14. Alternatively, the HMD may identify one or more anchors (not shown) being marked on the three-dimensional object such that the 3D image/model 204 may be aligned exactly with the object in certain display mode, for example, the 3D image 204 may be automatically position at a fixed distance from the ankle 1402 of the patient 1404 when being viewed by the surgeon wearing the HMD, so that the 3D image 204 is close to a focus of the surgeon but it does not obstruct a view of the ankle 1402 during a surgical operation.

In this example, the 3D model 204 of the ankle 1402 is display on the left side of the AR/MR display, while the original 2D CT/MRI images 202/202A are shown on the right side of the AR/MR display. Preferably, when the surgeon moves the section plane to change to a sectional view of the 3D model 204, the corresponding source 2D CT/MRI (raw) image 202/202A is shown on the right side for being observed by the surgeon simultaneously, without requiring the surgeon to manually choose the correct 2D image to be opened by a separate DICOM viewer.

These embodiments may be advantageous in that a 3D image processing pipeline is provided with a 2D pre-processing module suitable for pre-processing multiple sets of raw images in a batch and automated approach using the high performance image processing pipeline, which may effectively eliminate, or at least reduce, manual inputs for optimizing typical medical diagnostic scanning images such as CT, MRI, PET and X-ray scans before the images may be used as source images for 3D model construction based on segmentation and 3D mesh points extracted from these source images. The processing pipeline also capable of taking manual inputs which may further improve the 3D image produced according to the preference of the surgeon if necessary.

In addition, the constructed 3D image is embedded with the source 2D scans which allow the clinician or the surgeon to review the original 2D whenever necessary, and simultaneously with the 3D model showing the corresponding cross-sectional view, as experienced surgeons or practitioners may be used to viewing 2D cross-sectional scans using conventional DICOM viewer therefore displaying matching 2D source images and 3D cross-section views simultaneously may be preferred by some practitioners. By providing the 2D/3D image mapping data, additional operations to select/open a desired 2D scan image are no longer necessary as the matching 2D scanning image will be displayed automatically simply by manipulating the 3D model/image in a sectional viewing mode. Preferably, in this implementation, the 2D scan images are the sprites within the sprite sheets as created in step (318), and together would present the different sprites or images of the body part, tissue, bone etc as concerned in multiple cross sections. Therefore, when the surgeon or user “zooms in and out” of the cross sections, the sprites could therefore be presented frame by frame to animate the “zooming in and out” action by the surgeon to show the cross sections of the body part, tissue, bone etc of concern and thus allowing the surgeon, or other user, to access the cross sectional images from the 3D model with relative ease and intuition in their manipulation of the 3D model.

Although not required, the embodiments described with reference to the Figures can be implemented as an application programming interface (API) or as a series of libraries for use by a developer or can be included within another software application, such as a terminal or personal computer operating system or a portable computing device operating system. Generally, as program modules include routines, programs, objects, components and data files assisting in the performance of particular functions, the skilled person will understand that the functionality of the software application may be distributed across a number of routines, objects or components to achieve the same functionality desired herein.

It will also be appreciated that where the methods and systems of the present invention are either wholly implemented by computing system or partly implemented by computing systems then any appropriate computing system architecture may be utilised. This will include standalone computers, network computers and dedicated hardware devices. Where the terms “computing system” and “computing device” are used, these terms are intended to cover any appropriate arrangement of computer hardware capable of implementing the function described.

It will be appreciated by persons skilled in the art that numerous variations and/or modifications may be made to the invention as shown in the specific embodiments without departing from the spirit or scope of the invention as broadly described. The present embodiments are, therefore, to be considered in all respects as illustrative and not restrictive.

Any reference to prior art contained herein is not to be taken as an admission that the information is common general knowledge, unless otherwise indicated.

1. A method of three-dimensional image processing, comprising the steps of:
pre-processing at least one set of raw images each including a plurality of two-dimensional source images, wherein each of the two-dimensional source image represents a cross-sectional view of a three-dimensional object at different positions along an axis in a three-dimensional space; and
constructing a three-dimensional image representing the three-dimensional object by integrating 3D mesh points extracted from the at least one set of raw images being pre-processed;
wherein the three-dimensional image is readable by a first image viewer arrange to render and to facilitate manipulation of the three-dimensional image.

2. The method of claim 1, wherein the plurality of two-dimensional source images includes medical diagnostic scanning images.

3. The method of claim 2, wherein each of the at least one set of raw images includes a plurality of medical diagnostic scanning images obtained by a selected one of Computed Tomography (CT), Magnetic Resonance Imaging (MRI), Positron Emission Tomograph (PET) and x-ray imaging.

4. The method of claim 2, wherein the medical diagnostic scanning images are readable by a second image viewer.

5. The method of claim 4, wherein the step of constructing the three-dimensional image comprises the step of embedding the plurality of two-dimensional source images in the three-dimensional image at corresponding positions along the axis.

6. The method of claim 5, wherein the first image viewer is arranged to render a cross-sectional view of the three-dimensional object embedding with a two-dimensional view of the cross-section reproduced based on the medical diagnostic scanning image captured at the corresponding position.

7. The method of claim 6, wherein the two-dimensional view of the cross-section is substantially equal to the corresponding medical diagnostic scanning image read by the second image viewer.

8. The method of claim 1, further comprising the step of mapping the plurality of two-dimensional source images and the positions of the corresponding cross-section in the three-dimensional object along the axis.

9. The method of claim 1, further comprising the step of identifying and segmenting a plurality of components of different attributes or properties in the three-dimensional object.

10. The method of claim 9, wherein the plurality of components includes bone, soft tissue, fluid or an implant.

11. The method of claim 10, wherein the three-dimensional object includes at least a portion of an organ, one or more organ, or a combination thereof, of a living organism.

12. The method of claim 9, wherein the step of identifying the plurality of components comprises the step of facilitating manually masking of one or more of the plurality of components.

13. The method of claim 9, wherein the step of pre-processing the at least one set of raw images comprises the step of performing an image optimization process to optimize the at least one set of raw images prior to the step of identifying and segmenting the plurality of components.

14. The method of claim 13, wherein the image optimization process includes an image fusion process arranged to fuse the plurality of two-dimensional source images obtained in different sets of raw images.

15. The method of claim 14, wherein the image optimization process includes an interpolation process arranged to generate one or more augment images representing the cross-sectional view of the three-dimensional object at a position between two adjacent two-dimensional source images in the corresponding set of raw images captured along an axis.

16. The method of claim 3, further comprising the step of identifying and segmenting different set of raw images obtained by different medical diagnostic scanning methods.

17. A method of rendering a three-dimensional image constructed by the method of claim 1, comprising the step of rendering the three-dimensional image on a display module, wherein the display module is provided with a user interface arranged to facilitate manipulation of the three-dimensional image.

18. The method of claim 17, wherein the manipulation of the three-dimensional image includes at least one of zooming, rotating, translating the three-dimensional image and/or moving a section plane for sectioning the three-dimensional image along the axis.

19. The method of claim 17, wherein the step of rendering the three-dimensional image includes overlaying the three-dimensional image on the three-dimensional object when being observed by a user of the display module.

20. The method of claim 19, wherein the step of rendering the three-dimensional image further comprises the step of identifying one or more anchors being marked on the three-dimensional object.

21-30. (canceled)
